(19) United States
(12) Patent Application Publication
(10) Pub. No.: US 2020/0121198 A1
(43) Pub. Date: Apr. 23, 2020

(54) MULTI-PARAMETER VITAL SIGNS MONITORING DEVICE FOR EARLY WARNING SCORE SYSTEM

(71) Applicant: Fu-Chiang CHOU, Taoyuan City (TW)

(72) Inventor: Fu-Chiang CHOU, Taoyuan City (TW)

(21) Appl. No.: 16/655,846

(22) Filed: Oct. 17, 2019

Related U.S. Application Data
(60) Provisional application No. 62/746,568, filed on Oct. 17, 2018.

(51) Int. Cl. A61B 5/0205; A61B 5/1455; A61B 5/00; A61B 5/01

(52) U.S. Cl. CPC A61B 5/02055; A61B 5/14552; A61B 5/6802; A61B 5/01; A61B 5/02233; A61B 5/742; A61B 5/7455; A61B 5/4809; A61B 5/7405

(57) ABSTRACT

The present invention relates to wearable devices for monitoring of vital signs. More specifically, the present invention relates to a one-piece wearable device for mounting on the upper arm of the patient and monitoring of multi-parameter vital signs without any attached cable.

### BACKGROUND OF THE INVENTION

### 1. Field of the Invention

The present invention relates to wearable devices for monitoring of vital signs. More specifically, the present invention relates to a one-piece wearable device for mounting on the upper arm of the patient and monitoring of multi-parameter vital signs without any attached cable.

### 2. Description of the Related Art

Conventional vital sign monitors are used throughout the hospital, especially in high-acuity areas such as the Intensive Care Unit (ICU), or Operating Room (OR). However, it's more common to have an early warning score system in general wards to determine the degree of illness of the patients and prompts critical care intervention. The early warning score is calculated by the sum of the scores for individual vital signs such as blood pressure, respiratory rate, oxygen saturation (SpO2), heart rate, body temperature, and conscious level, etc. The monitoring of these parameters is typically done with portable or wall-mounted vital signs monitors. This poses a problem for conventional vital sign monitors, which are typically heavy and unwieldy, as they are not intended for the general inpatient population. Some companies had developed wearable vital signs monitors to collect multi-parameter vital signs automatically. However, because of the limitation of the methods for monitoring of multi-parameter vital signs, it needs multi-pieces of devices to monitor different vital signs at different positions of the body. For example, as shown in FIG. 3A and FIG. 3B of the U.S. Pat. No. 8,475,370B2, SpO2 is measured from the finger, the blood pressure is measured from the upper arm, and the ECG is measured by the patches on the chest. It's not easy for the nurse to put all these devices on the patients. It's not comfortable for the patients to wear many devices as well.

Another problem with conventional wearable vital signs monitors for measuring body temperature is that they are measuring the temperature on different positions on the skin that can't reflect the real body temperature. The best place to measure the body temperature in a continuous way should be under the armpit. The other places such as in the mouth, in the ear or in the rectum are not suitable for continuous measurement.

Another parameter like the conscious level that can't be measured directly from the vital signs was designed in some early warning score systems like the National Early Warning Score (NEWS) from the Royal College of Physicians. In order to measure this parameter, the nurse needs to interact with the patients to identify the conscious level of the patients. This makes it more difficult to have the early warning score automatically calculated by the conventional vital signs monitoring devices.

To sum up, it's necessary to develop a one-for-all device adaptable for measuring multi-parameter vital signs of the early warning score system. Moreover, it's crucial to propose a practical and progressive solution for continuous monitoring of the body temperature and the conscious level for inpatient nursing.

### SUMMARY OF THE INVENTION

The purpose of the invention is to make a wearable device that can measure 5 vital signs, including blood pressure, respiratory rate, SpO2, heart rate and body temperature in the same time. In addition, we also want to interact with the patient to identify his conscious level in a natural way.

The main body of the device is a blood pressure monitor that using the conventional oscillometric method and measure the blood pressure on the upper arm. The electrical system and the cuff were integrated together without an additional air tube. In order to measure the SpO2 in the same place, a reflectance type of pulse oximetry sensor was integrated into the cuff. The heart rate and the respiratory rate can be derived from the Photoplethysmography (PPG) signals measured by the pulse oximetry sensor. The body temperature is measured by a thermal sensor integrated with the cuff under the armpit. Therefore, we can use this integrated device to measure the 5 parameters of the vital signs, blood pressure, SpO2, heart rate, respiratory rate, and body temperature.

A software process installed in the device will interact with the patient to get his conscious level. This process is combined with the process of measuring blood pressure in order to make the whole procedure intuitively. When the system is trying to measure the blood pressure, it will prompt the patient with hints (with sounds, vibration, or light) to press the push-button on the main body to start the measuring of the blood pressure. If the patient does react properly, that means the conscious level of the patient is normal. If the patient doesn't react properly, that means the patient may be unconscious. This procedure can be used to decide the conscious level of the patient.

### BRIEF DESCRIPTION OF THE DRAWINGS

FIG. 1 is an exterior view of the embodiment to show the cuff and thermal sensor for body temperature.

FIG. 2 is another perspective view of the embodiment to show the reflectance pulse oximetry sensor for SpO2.

FIG. 3 is the system diagram of the embodiment to show the necessary components of the device.

### DETAILED DESCRIPTION OF PREFERRED EMBODIMENTS

One embodiment of the invention is described more fully hereinafter with reference to the accompanying drawings, in which not all embodiments of the invention are shown in the figures.

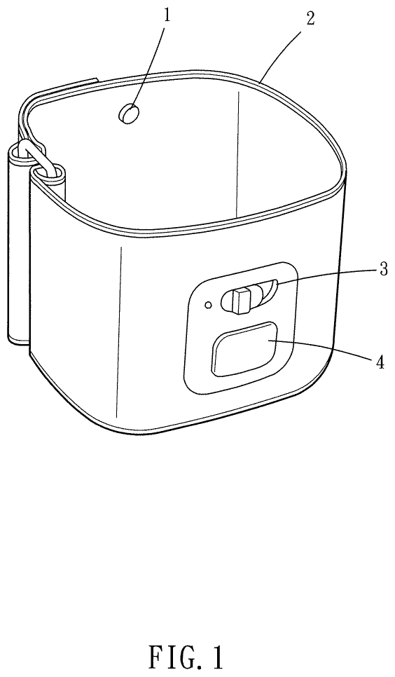

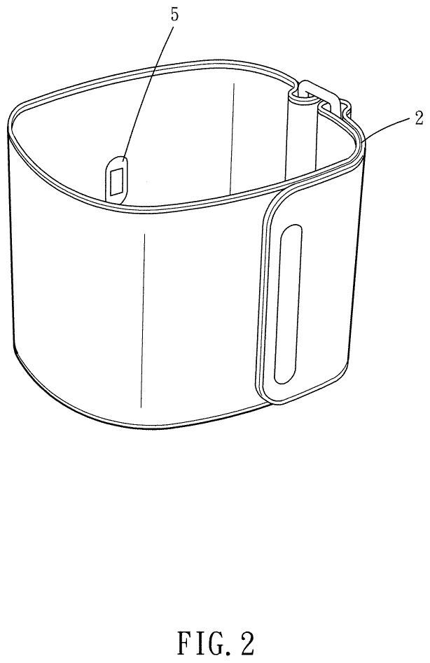

FIG. 1 illustrates a one-piece wearable device of the embodiment. As shown, a thermal sensor 1 is integrated with the cuff 2 of the blood pressure monitor. Because the cuff 2 of the blood pressure monitor will be put on the upper arm of the user, the thermal sensor, therefore, will be placed on the armpit when the user wears the device. With this design, the thermal sensor 1 can measure the temperature on the armpit that reflects one of the standard positions for measuring the body temperature. Button switch 3 is used to turn on/off the wearable device. Push-button 4 is used to start or stop the measuring process of the blood pressure. When the device prompts the user to start the measuring of the blood pressure, the user needs to push the button to start the process. Thus, the device will identify the conscious level of the user according to the user's behavior of the interaction. FIG. 2 illustrates another view of the wearable device to show the reflectance pulse oximetry sensor 5 which is integrated with the cuff 2. There is a hole in the cuff 2 so the light of the pulse oximetry sensor can be radiated on the skin and reflected into the photodetector in the sensor to measure the SpO2. As shown in FIG. 1 and FIG. 2, the reflectance pulse oximetry sensor 5 is opposite to the thermal sensor 1, but it's not a restriction.

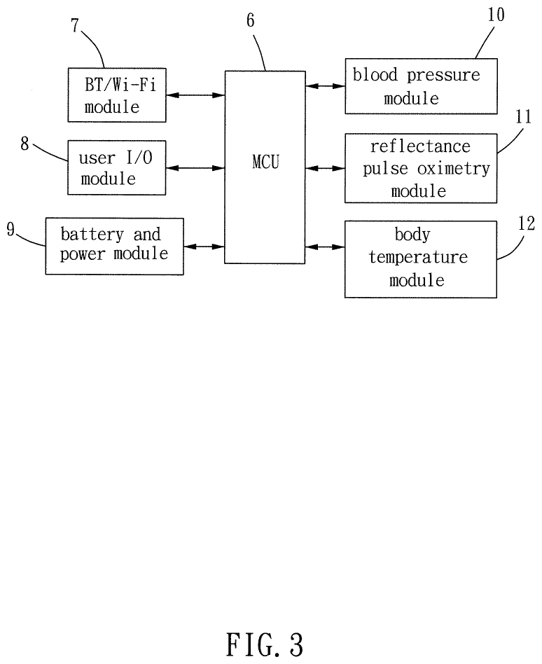

FIG. 3 illustrates the system diagram of the wearable device. MCU 6 is used to control the whole system. The BT & Wi-Fi module 7 is used to build a connection between the wearable device and other equipment, so the data collected from the device can be transmitted for further usage. The user I/O module 8 (having the push-button 4) is designed to interact with the user by sound, vibration or light according to the scenarios. The battery and power module 9 is designed to provide the power for the system and is connected to the MCU 6. The blood pressure module 10 is used to measure the blood pressure and send the data to MCU 6. The reflectance pulse oximetry module 11 (having the reflectance pulse oximetry sensor 5 and other components) is used to measure the Photoplethysmography (PPG) signals and send the data to MCU 6. This data is used for the calculation of SpO2, heart rate and respiratory rate by corresponding algorithms. The body temperature module 12 (having the thermal sensor 1 and other components) is used to measure the body temperature and send the data to MCU 6.

The embodiment of the invention is described in the following description: A wearable vital signs monitoring device including a blood pressure monitor having an integrated cuff 2; a reflectance pulse oximetry having the SpO2 measuring ability on the upper arm, and the reflectance pulse oximetry sensor 5 is integrated with cuff 2 and measuring the SpO2 through a hole on cuff 2; a thermal sensor 1 having the measuring ability of the body temperature on the armpit, which is also attached on the cuff 2. Said wearable vital signs monitoring device further including a push-button 4, wherein the device can prompt the user to start the measurement of the blood pressure by pressing the push-button 4 and the system will determine the conscious level according to the user's interaction. Said wearable vital signs monitoring device further including a Bluetooth & Wi-Fi module 7, used to build a connection between the device and other equipment so the data collected from the device can be transmitted for further usage; user I/O module 8, designed to interact with the user by sound, vibration, or light according to the scenarios.

In other embodiments, said wearable vital signs monitoring device may provide fall detection, positioning system, or user identification function for safety and security purposes.

The implementation of the invention is to modify the conventional methods of measuring different vital signs from different positions by integrating the device into one-piece and measure different vital signs in one single position to solve the problems of inconvenience and uncomfortableness. The invention measures the real body temperature from the armpit instead of the skin temperature from other positions. Moreover, the invention discloses a method to identify the conscious level that can't be directly calculated from the vital signs with an intuitive interaction process. This invention can be used for the early warning score system of inpatient nursing in the hospital. The Bluetooth & Wi-Fi module 7 can transmit the collected vital signs data to the Hospital Information System (HIS) for further usage. This information is crucial in order to provide mandatory nursing care to patients.

### DETAILED DESCRIPTION OF THE COMPONENTS IN THE DRAWINGS

1 thermal sensor
2 cuff
3 button switch
4 push-button
5 reflectance pulse oximetry sensor
6 MCU
7 BT/Wi-Fi module
8 user I/O module
9 battery and power module
10 blood pressure module
11 reflectance pulse oximetry module
12 body temperature module

What is claimed is:

1. A one-piece wearable vital signs monitoring device, comprising:
a blood pressure monitor having a cuff;
a reflectance pulse oximetry sensor mounted inside the cuff, and the optical element of the reflectance pulse oximetry sensor attached inside the cuff for measuring pulse oximetry signals from the user; and said wearable vital signs monitoring device is designed at least for measuring blood pressure, pulse oximetry and heart rate.

2. A wearable vital signs monitoring device as claimed in claim 1, said wearable vital signs monitoring device further including a microprocessor for deriving a respiratory rate from the Photoplethysmography (PPG) signals measured by the pulse oximetry sensor.

3. A wearable vital signs monitoring device as claimed in claim 1, said wearable vital signs monitoring device further including a thermal sensor that is attached inside the cuff for measuring the body temperature on the armpit.

4. A wearable vital signs monitoring device as claimed in claim 1, said wearable vital signs monitoring device will measure the blood pressure periodically by prompting the user with a sound, vibration, or light as a hint to start the measurement of the blood pressure, then determine the user's conscious level by the corresponding feedback of the user.

5. A wearable vital signs monitoring device as claimed in claim 2, said wearable vital signs monitoring device further including a thermal sensor that is attached inside the cuff for measuring the body temperature on the armpit.

6. A wearable vital signs monitoring device as claimed in claim 2, said wearable vital signs monitoring device will measure the blood pressure periodically by prompting the user with a sound, vibration, or light as a hint to start the measurement of the blood pressure, then determine the user's conscious level by the corresponding feedback of the user.

7. A wearable vital signs monitoring device as claimed in claim 3, said wearable vital signs monitoring device will measure the blood pressure periodically by prompting the user with a sound, vibration, or light as a hint to start the measurement of the blood pressure, then determine the user's conscious level by the corresponding feedback of the user.

8. A wearable vital signs monitoring device as claimed in claim 5, said wearable vital signs monitoring device will measure the blood pressure periodically by prompting the user with a sound, vibration, or light as a hint to start the measurement of the blood pressure, then determine the user's conscious level by the corresponding feedback of the user.
